(19) United States
(12) Patent Application Publication
(10) Pub. No.: US 2023/0000351 A1
(43) Pub. Date: Jan. 5, 2023

(54) Monitoring System, Monitoring Method, and Monitoring Program

(71) Applicant: Nippon Telegraph and Telephone Corporation, Tokyo (JP)

(72) Inventors: Takayuki Ogasawara, Tokyo (JP); Kenichi Matsunaga, Tokyo (JP); Rieko Sato, Tokyo (JP)

(21) Appl. No.: 17/779,857

(22) PCT Filed: Dec. 5, 2019
(86) PCT No.: PCT/JP2019/047634

§ 371 Date: May 25, 2022

(51) Int. Cl. A61B 5/00; G16H 40/67

(52) U.S. Cl. CPC A61B 5/002; G16H 40/67; A61B 5/0004

(57) ABSTRACT

A monitoring system includes a first acquisition unit that acquires identification information unique to a user, a second acquisition unit that acquires position information of the user, an action history calculation unit that obtains an action history of the user from the identification information of the user acquired by the first acquisition unit and the position information acquired by the second acquisition unit, and a presentation unit that presents the action history of the user calculated by the action history calculation unit, wherein the action history includes at least one of a period for which and a frequency at which the user stayed at a position indicated by the position information.

### CROSS-REFERENCE TO RELATED APPLICATIONS

This patent application is a national phase filing under section 371 of PCT application no. PCT/JP2019/047634, filed on Dec. 5, 2019, which application is hereby incorporated herein by reference in its entirety.

### TECHNICAL FIELD

The present invention relates to a monitoring system, a monitoring method, and a monitoring program, and particularly to a technique for watching over a patient in medical care and long-term care.

### BACKGROUND

Conventionally, there has been proposed a monitoring system that enables monitoring of a patient using a sensor, and focuses on the daily life rhythm of the patient for medical care and long-term care facilities (see Non-Patent Literature 1). FIG. 16 is a diagram showing an overview of the conventional monitoring system disclosed in Non-Patent Literature 1.

As shown in FIG. 16, in the conventional monitoring system, a user such as a patient wears rehabilitation wear that is a wearable device, and data of the electrocardiographic potential and acceleration of the user for 24 hours is acquired by the wearable device. The rehabilitation wear is provided with a transmitter, and information on the electrocardiographic potential and acceleration of the user is transmitted from the transmitter to a relay terminal device such as a smartphone or an IoT gate.

The data of the electrocardiographic potential and acceleration of the user is subjected to data storage, accumulation, and analysis processing in an external terminal device such as a server connected via a network. The analysis result is output based on biological information of the user analyzed in the external terminal device, and is notified to medical personnel responsible for the medical care and nursing care for the user, such as doctors, therapists, and nurses, through a viewer.

From the notified analysis result and report, the doctors, therapists, nurses, and others can provide more suitable care to the user when treating or caring for the user whom they are responsible for.

However, information obtained from the electrocardiographic potential and acceleration information of the user for 24 hours in the conventional monitoring system described in Non-Patent Literature 1 is a measurement result of sensor data, and its typical content is information indicating that the user's posture was a lying posture and the heart rate decreased. Even though such a change in the user's posture and heart rate indicates abnormality in biological information and activity information of the user, it does not directly indicate the cause of the abnormality, so, for example, it may be difficult to provide appropriate guidance in life to users whose amount of activity is low.

The activity of a user such as a patient is often determined by the user's whereabouts as their living environment. For example, if the user is led to spend their time in a small hospital room, they have no choice but to spend most of their time lying down or sitting on the bed or the like. In such a case, the user's posture is often a lying posture, and the heart rate also decreases, which are as indicated by the biological information and activity information of the user obtained by the conventional monitoring system.

### CITATION LIST

### Non-Patent Literature

Non-Patent Literature 1: Ogasawara Takayuki, Matsunaga Kenichi, Ito Hiroki, Oshima Shoichi, Mukaino Masahiko, “Efforts to Support Rehabilitation by Applying Wearable Material hitoe (R)”, NIT Technical Journal 2018.7 (pp. 10-14, FIG. 3).

### SUMMARY

### Technical Problem

If the action history of the user such as where, how often, and how much time the user spent can be grasped by grasping the user's whereabouts, it becomes possible to support improvement of the user's life more concretely and appropriately when trying to increase the amount of activity of the user, for example.

Embodiments of the present invention has been made to solve the above problems, and aims to grasp the action history of a user.

### Means for Solving the Problem

In order to solve the above problems, a monitoring system according to embodiments of the present invention includes: a first acquisition unit that acquires identification information unique to a user; a second acquisition unit that acquires position information of the user; a calculation unit that obtains an action history of the user from the identification information of the user acquired by the first acquisition unit and the position information acquired by the second acquisition unit; and a presentation unit that presents the action history of the user calculated by the calculation unit, wherein the action history includes at least one of a period for which and a frequency at which the user stayed at a position indicated by the position information.

In order to solve the above problems, a monitoring system according to embodiments of the present invention includes: a sensor terminal device that is attached to a user, and outputs first identification information, which is identification information unique to the sensor terminal device, to outside; a relay terminal device that is arranged at a predetermined position within an area, receives the first identification information output from the sensor terminal device, and outputs the first identification information and second identification information, which is identification information unique to the relay terminal device, to outside; and an external terminal device that receives the first identification information and the second identification information output from the relay terminal device and stores the first identification information and the second identification information in a storage device, wherein the external terminal device includes a first acquisition unit that acquires the first identification information as identification information unique to the user, a second acquisition unit that acquires the second identification information as position information of the user, a calculation unit that obtains an action history of the user from the identification information of the user acquired by the first acquisition unit and the position information acquired by the second acquisition unit, and a presentation unit that presents the action history of the user obtained by the calculation unit, and the action history includes at least one of a period for which and a frequency at which the user stayed at a position indicated by the position information.

In order to solve the above problems, a monitoring method according to embodiments of the present invention includes: a first step of acquiring identification information unique to a user; a second step of acquiring position information of the user; a third step of obtaining an action history of the user from the identification information of the user acquired in the first step and the position information acquired in the second step; and a fourth step of presenting the action history of the user calculated in the third step, wherein the action history includes at least one of a period for which and a frequency at which the user stayed at a position indicated by the position information.

In order to solve the above problems, a monitoring program according to embodiments of the present invention causes a computer to execute the above monitoring method.

### Effects of Embodiments of the Invention

According to embodiments of the present invention, since an action history of a user is obtained and presented from identification information unique to the user acquired by the first acquisition unit, and position information of the user acquired by the second acquisition unit, it is possible to grasp the action history of the user.

### BRIEF DESCRIPTION OF THE DRAWINGS

FIG. 1 is a block diagram showing a functional configuration of a monitoring system according to a first embodiment of the present invention.

FIG. 2 is a block diagram showing an example of a computer configuration that implements the monitoring system according to the first embodiment.

FIG. 3 is a flowchart for describing a monitoring method according to the first embodiment.

FIG. 4 is a diagram for describing an interpolation unit according to the first embodiment.

FIG. 5 is a diagram for describing an overview of an example configuration of the monitoring system according to the first embodiment.

FIG. 6 is a block diagram showing an example configuration of the monitoring system according to the first embodiment.

FIG. 7 is a block diagram showing a configuration of a monitoring system according to a second embodiment.

FIG. 8 is a schematic diagram for describing operation of the monitoring system according to the second embodiment.

FIG. 9 is a diagram for describing an interpolation unit according to the second embodiment.

FIG. 10 is a diagram for describing the interpolation unit according to the second embodiment.

FIG. 11 is a block diagram showing a configuration of a monitoring system according to a third embodiment.

FIG. 12 is a flowchart showing a monitoring method according to the third embodiment.

FIG. 13 is a diagram for describing an estimation unit according to the third embodiment.

FIG. 14 is a diagram for describing the estimation unit according to the third embodiment.

FIG. 15 is a block diagram showing an example configuration of the monitoring system according to the third embodiment.

FIG. 16 is a diagram for describing an overview of a conventional monitoring system.

### DETAILED DESCRIPTION OF ILLUSTRATIVE EMBODIMENTS

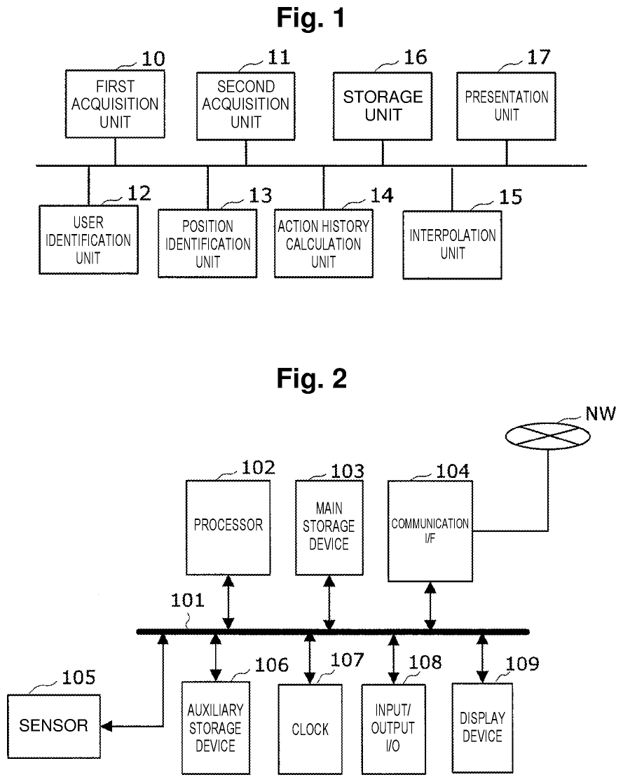

Hereinafter, preferred embodiments of the present invention will be described in detail with reference to FIGS. 1 to 15.

### Overview of Embodiments of the Invention

First, an overview of monitoring systems according to the embodiments of the present invention will be described. The monitoring systems according to the embodiments identify individual users such as users who perform rehabilitation in a long-term care facility and hospitalized patients, and also identify the position of each user in the facility. In addition, the monitoring systems according to the embodiments calculate an action history of the user including the staying time of the user at the identified position. Furthermore, when a period in which the position of the user cannot be identified has occurred and data of the action history has been lost, the monitoring systems according to the embodiments interpolate the action history of the user from identification information and position information of the user acquired before and after the lost period.

### First Embodiment

First, an overview of a configuration of a monitoring system according to a first embodiment of the present invention will be described. FIG. 1 is a block diagram showing a functional configuration of the monitoring system.

Functional Blocks of Monitoring System

The monitoring system includes a first acquisition unit 10, a second acquisition unit 11, a user identification unit 12, a position identification unit 13, an action history calculation unit (calculation unit) 14, an interpolation unit 15, a storage unit 16, and a presentation unit 17.

The first acquisition unit 10 acquires identification information unique to a user. For example, from a tag or a sensor terminal device 200 described later attached to the user, the first acquisition unit 10 acquires identification information of the device such as a MAC address, an IP address, or a serial number assigned to the sensor terminal device 200 as identification information of the user. The identification information of the device attached to the user such as the tag or the sensor terminal device 200 and the identification information of the user are stored in advance in the storage unit 16 in association with each other.

The second acquisition unit 11 acquires position information of the user. For example, the second acquisition unit 11 acquires identification information of a point arranged at a determined position within a facility or unique identification information owned by a relay terminal device 300 described later arranged within the facility as the position information of the user.

The user identification unit 12 identifies an individual user from the identification information unique to the user that is acquired by the first acquisition unit 10. The user identification unit 12 refers to the storage unit 16, and identifies the user corresponding to the identification information acquired by the first acquisition unit 10.

The position identification unit 13 identifies the position of the user from the position information acquired by the second acquisition unit 11. The position identification unit 13 identifies the position of the user at a certain cycle, and the position identification unit 13 outputs the identified position of the user for each time. For example, the position information in the facility and the identification information of the point or the relay terminal device 300 are stored in advance in the storage unit 16 in association with each other. The position identification unit 13 can refer to the storage unit 16, and identify the position in the facility that is linked to the position information acquired by the second acquisition unit 11, such as a “rehabilitation room” or a “cafeteria”.

The action history calculation unit 14 obtains an action history of the user from the user and the position of the user identified by the user identification unit 12 and the position identification unit 13. The action history is information on the position of the user in the facility according to the passage of time. The action history includes the period for which and the frequency at which the user stayed at the position identified by the position identification unit 13. For example, the action history calculation unit 14 can output information indicating that a “user A” stayed at a “cafeteria” once for one hour as the action history.

In addition, the action history calculation unit 14 can also obtain a time series of positions representing movements of the user in the facility, in addition to the period for which and the frequency at which they stayed at a specific position in the facility. The action history calculation unit 14 obtains the action history of the user at a certain cycle. For example, the action history of the user can be updated according to the cycle at which the second acquisition unit 11 acquires the position information of the user. The action history of the user obtained by the action history calculation unit 14 is stored in the storage unit 16.

When the action history of the user calculated by the action history calculation unit 14 includes a data loss period, the interpolation unit 15 confirms whether the pieces of position information of the user immediately before and after the loss period match each other. When the pieces of position information of the user immediately before and after the loss period included in the action history match each other, the interpolation unit 15 interpolates data of the action history of the user using the pieces of position information immediately therebefore and thereafter.

As described before, the action history calculation unit 14 cannot obtain the action history of the user unless both the identification information unique to the user and the position information are acquired. When the action history calculation unit 14 cannot obtain the action history of the user in a period of time, the time series of the action history of the user will include a loss period.

For example, suppose that the actual position of the user is the same throughout one hour, but two momentary losses have occurred in the action history of the user for one hour, and there have occurred loss periods during which the identification information and the position information of the user cannot be acquired. Originally, the fact that the user stayed at the same position at a frequency of once for about one hour should be obtained as the action history. However, when loss periods have occurred in the action history, the action history calculation unit 14 incorrectly calculates that the user stayed at the same place at a frequency of three times and stayed for a period shorter than one hour when obtaining the staying frequency (the number of stays) of the user, though it is originally once.

When a loss period has occurred in the action history of the user, the interpolation unit 15 detects the loss period, and, when the user and the position of the user identified by the user identification unit 12 and the position identification unit 13 before and after the loss period match each other, interpolates the action history by assuming that the position of the user during the loss period did not change before and after the loss period and during the loss period.

The storage unit 16 stores the identification information unique to the user. The storage unit 16 stores, for example, the user's name or ID number, the identification information unique to the device, such as a MAC address, an IP address, or a serial number assigned in advance to the device, carried and moved by the user, such as the sensor terminal device 200 assigned to the user, and user information in association with each other.

Further, the storage unit 16 stores identification information of a device from which the position information of the user is acquired, such as identification information of a point arranged in the facility or identification information such as a MAC address or an IP address of the relay terminal device 300 described later, and information indicating the position where the device is arranged in the facility in association with each other. For example, the position coordinate at which the relay terminal device 300 having a predetermined communication area is installed in the long-term care facility, or the name of a room covered by the communication area, such as “cafeteria”, “entrance”, or “washroom”, and identification information, such as a MAC address, and position information of the relay terminal device 300 are stored in association with each other.

Further, the storage unit 16 stores the action history of the user obtained by the action history calculation unit 14. The action history of the user is, for example, data indicating a time series of position information and a staying time and staying frequency at each position for each user.

The presentation unit 17 presents the action history of the user obtained by the action history calculation unit 14. For example, the presentation unit 17 can display the action history of the user on a display screen of a display device 109 described later.

Hardware Configuration of Monitoring System

Next, an example of a computer configuration that implements the monitoring system having the above functions will be described with reference to FIG. 2.

As shown in FIG. 2, the monitoring system can be implemented by, for example, a computer including a processor 102, a main storage device 103, a communication I/F 104, an auxiliary storage device 106, a clock 107, and an input/output I/O 108 connected via a bus 101, and a program that controls these hardware resources. For example, each of a sensor 105 and a display device 109 that are provided externally is connected to the monitoring system via the bus 101.

The main storage device 103 stores in advance programs for the processor 102 to perform various types of control and calculation. The processor 102 and the main storage device 103 implement the functions of the monitoring system including the user identification unit 12, the position identification unit 13, the action history calculation unit 14, and the interpolation unit 15 shown in FIG. 1.

The communication I/F 104 is an interface circuit for communicating with various types of external electronic equipment via a communication network NW.

As the communication I/F 104, for example, a communication control circuit and an antenna supporting wireless data communication standards such as 3G, 4G, 5G, wireless LAN, Bluetooth (R), and Bluetooth Low Energy are used. The communication I/F 104 implements the first acquisition unit 10 and the second acquisition unit 11 described in FIG. 1.

The sensor 105 includes, for example, an electrocardiograph and a triaxial acceleration sensor. The sensor 105 can further include, for example, a sensor for measuring biological information and physical information of the user such as a sphygmomanometer, a pulse meter, a respiration sensor, a thermometer, and a brain wave sensor. When the presentation unit 17 described in FIG. 1 displays the action history of the user on the display screen, a time series of biological information of the user measured by the sensor 105 can be displayed together with the action history.

The auxiliary storage device 106 includes a readable and writable storage medium and a driving device for reading/writing various types of information such as programs and data from/to the storage medium. For the auxiliary storage device 106, a semiconductor memory such as a hard disk or a flash memory can be used as a storage medium.

The auxiliary storage device 106 has a program storage area that stores programs for the monitoring system to calculate the action history and perform interpolation processing of data of the action history and monitoring programs. The auxiliary storage device 106 implements the storage unit 16 described in FIG. 1. The auxiliary storage device 106 may have a storage area for storing biological information of the user measured by the sensor 105, and also, for example, a backup area for backing up the above-mentioned data and programs.

The clock 107 includes an internal clock built in the computer or the like, and measures the time. Alternatively, the clock 107 may acquire time information from a time server not shown.

The input/output I/O 108 includes an I/O terminal that receives a signal from external equipment as input, and outputs a signal to external equipment.

The display device 109 is implemented by a liquid crystal display or the like. The display device 109 implements the presentation unit 17 in FIG. 1.

Monitoring Method

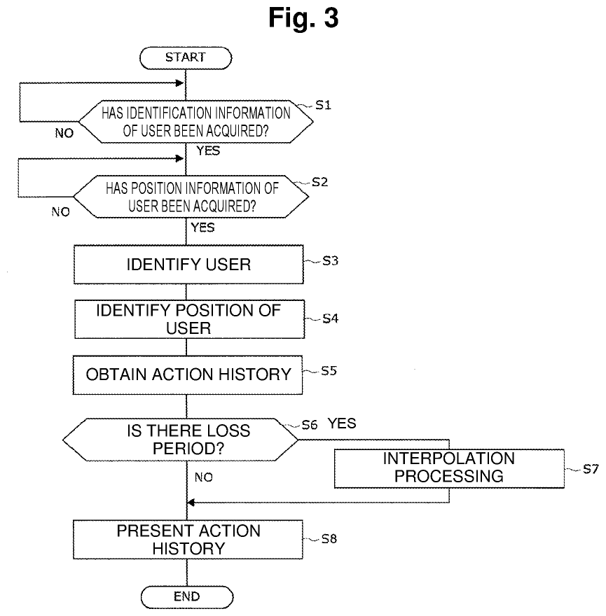

Next, operation of the monitoring system having the above-described configuration will be described using a flowchart in FIG. 3. In the following, it is assumed that the storage unit 16 stores user information (e.g., the user's name or patient ID) and identification information unique to a wearable device assigned to the user (e.g., a MAC address or an IP address) in association with each other. Further, it is assumed that the storage unit 16 stores unique identification information (e.g., a MAC address or an IP address) of a point arranged at a fixed position in the facility, the relay terminal device 300 or other devices, and information indicating the arrangement position (e.g., a name such as “cafeteria” or “entrance”) in association with each other.

As shown in FIG. 3, the first acquisition unit 10 first acquires identification information unique to the user (step S1). For example, the first acquisition unit 10 acquires unique identification information assigned to a wearable device attached to the user.

Next, the second acquisition unit 11 acquires position information of the user (step S2). For example, from a point or IoT gate in the facility that has established communication with the wearable devices worn by the user, the second acquisition unit 11 acquires unique identification information assigned to these devices. Further, the second acquisition unit 11 can acquire the position information of the user at a certain cycle.

Next, the user identification unit 12 identifies the user from the identification information of the user acquired by the first acquisition unit 10 (step S3). Next, the position identification unit 13 identifies the position of the user from the position information acquired by the second acquisition unit 11 (step S4). The user identification unit 12 and the position identification unit 13 identify the user and the position of the user from the information stored in advance in the storage unit 16.

Next, the action history calculation unit 14 obtains an action history of the user (step S5). For example, the action history calculation unit 14 calculates the frequency (the number of times) at which and the period for which the user stayed at the identified position in the facility.

Next, when the action history calculated by the action history calculation unit 14 includes a loss period (step S6: YES), the interpolation unit 15 performs interpolation processing (step S7). More specifically, the interpolation unit 15 detects that there is a loss period in the action history, and, when the position of the user identified in step S4 immediately before the loss period is the same as the position of the user identified in step S4 immediately after the loss period, regards the position information of the user during the loss period as the same as the pieces of position information immediately before and after the loss period.

After that, the presentation unit 17 displays the action history interpolated by the interpolation unit 15 on, for example, the display screen of the display device 109 (step S8). On the other hand, when a loss period in the action history is not detected in step S6 (step S6: NO), the interpolation processing by the interpolation unit 15 is not executed, and the action history of the user obtained in step S5 is presented by the presentation unit 17 (step S8). At this time, the presentation unit 17 can present the heart rate or the like of the user measured by the sensor 105 together with the action history of the user.

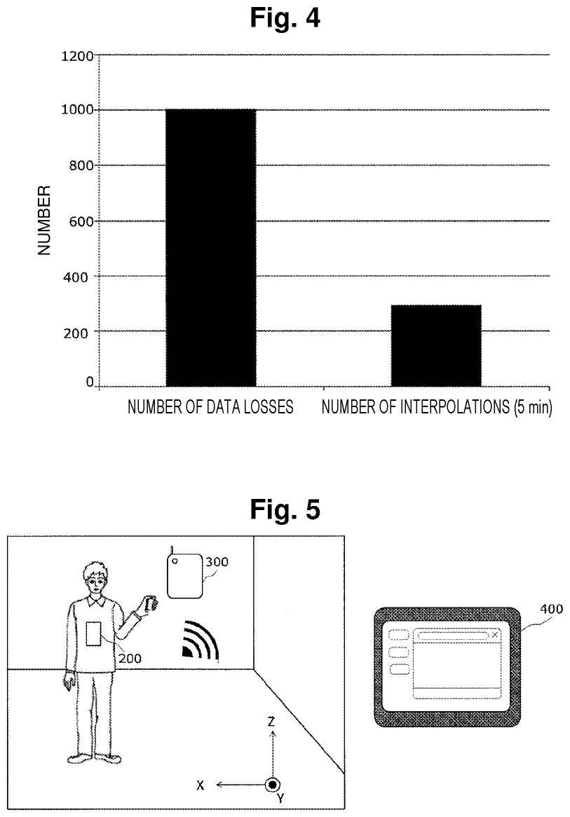

FIG. 4 is a graph showing the effect of the interpolation processing by the interpolation unit 15 according to this embodiment. The bar graph on the left side of FIG. 4 shows the number of data losses that have occurred in the action history in a certain period, and about 1,000 data losses have occurred intermittently. On the other hand, the bar graph on the right side of FIG. 4 shows the number of cases of interpolation processing when the interpolation unit 15 performed interpolation for data losses for a period of 5 minutes or less in the same period of time. From this, it can be seen that by the monitoring system being provided with the interpolation unit 15, the data is improved by about 300 pieces by the interpolation processing. Thus, the monitoring system according to this embodiment can obtain the action history of the user with higher reliability by having the interpolation unit 15.

Specific Configuration of Monitoring System

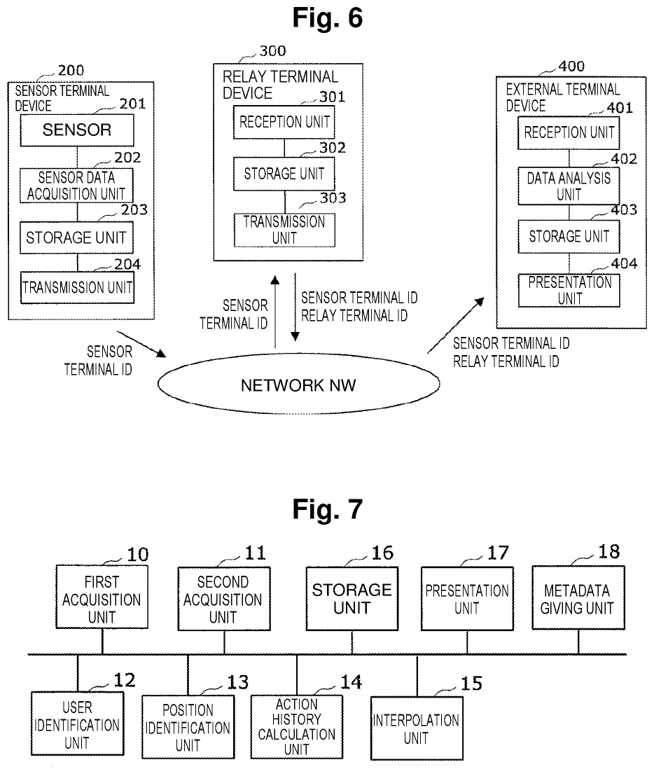

Next, an example specific configuration of the monitoring system having the above-described configuration will be described with reference to FIGS. 5 and 6. For example, as shown in FIG. 5, the monitoring system includes, for example, the sensor terminal device 200 attached to a user who performs rehabilitation, the relay terminal device 300, and an external terminal device 400.

The sensor terminal device 200 includes a wearable device or the like, and is attached to the user to move together with the user in a facility such as a rehabilitation facility. The sensor terminal device 200 has unique identification information, and the identification information of the sensor terminal device 200 makes it possible to identify which user the user is.

As the relay terminal device 300, for example, a smart phone, a tablet terminal, a laptop, and a small computer typified Raspberry Pi (R) and OpenBlocks (R) can be used. The relay terminal device 300 is arranged at a fixed position in a facility to be monitored. A plurality of relay terminal devices 300 are arranged in advance in the facility.

The relay terminal device 300 has its own communication area. When the sensor terminal device 200 attached to the user has entered the communication area of the relay terminal device 300, the sensor terminal device 200 permitted in advance to perform communication can perform wireless communication with the relay terminal device 300. The identification information unique to the relay terminal device 300 and the position information indicating the position where the relay terminal device 300 is arranged in the facility are registered in advance in association with each other. The identification information of the relay terminal device 300 makes it possible to identify the position information of the user.

As shown in FIG. 5, the relay terminal device 300 is arranged on the ceiling or a wall of a room in the facility. Further, it is assumed in this embodiment that the communication area of the relay terminal device 300 is treated as the position of one point in the facility.

As with the relay terminal device 300, as the external terminal device 400, for example, a smart phone, a tablet terminal, a laptop, and a small computer typified by Raspberry Pi (R) and OpenBlocks (R) are used.

The external terminal device 400 is provided with the functions of the monitoring system described in FIG. 1, and performs wired or wireless communication with the relay terminal device 300.

Configuration of Sensor Terminal Device

As shown in FIG. 6, the sensor terminal device 200 includes a sensor 201, a sensor data acquisition unit 202, a storage unit 203, and a transmission unit 204. The sensor terminal device 200 is, for example, arranged on the user's body trunk to move together with the user in the facility to be monitored. When the sensor terminal device 200 has entered the communication area of the relay terminal device 300, it establishes wireless communication with the relay terminal device 300, and transmits unique identification information such as a MAC address or an IP address assigned to the sensor terminal device 200.

The sensor 201 is implemented by, for example, an electrocardiograph and a triaxial acceleration sensor. For the three axes of the acceleration sensor provided in the sensor 201, for example, as shown in FIG. 5, the X-axis is provided in parallel with the left-right direction of the body, the Y-axis in the front-rear direction of the body, and the Z-axis in the up-down direction of the body. The sensor 201 corresponds to the sensor 105 described in FIG. 2.

The sensor data acquisition unit 202 acquires biological information of the user measured by the sensor 201. More specifically, the sensor data acquisition unit 202 performs noise removal and sampling processing for the acquired electrocardiographic potential, acceleration and the like to obtain time series of an electrocardiographic waveform, a heart rate, and acceleration in the form of digital signals.

The storage unit 203 stores the time-series data of the biological information of the user measured by the sensor 201. Further, the storage unit 203 stores the identification information of its own device. The storage unit 203 corresponds to the storage unit 16 (FIG. 1).

The transmission unit 204 transmits the biological information such as the heart rate of the user and the identification information (first identification information) of its own device that are stored in the storage unit 203 to the relay terminal device 300 in the communication area. The transmission unit 204 is provided with a communication circuit for performing wireless communication supporting wireless data communication standards such as LTE, 3G, 4G, 5G, wireless LAN (Local Area Network), Bluetooth (R), and Bluetooth Low Energy.

Configuration of Relay Terminal Device

The relay terminal device 300 includes a reception unit 301, a storage unit 302, and a transmission unit 303. The relay terminal device 300 transmits the identification information of the sensor terminal device 200 and the biological information of the user measured by the sensor terminal device 200 that are received from the sensor terminal device 200, and the identification information (second identification information) of the relay terminal device 300 to the external terminal device 400 via the communication network NW.

The reception unit 301 receives the identification information of the sensor terminal device 200 from the sensor terminal device 200 via the communication network NW.

The storage unit 302 stores the identification information of the sensor terminal device 200 received by the reception unit 301. Further, the storage unit 302 temporarily stores the biological information of the user measured by the sensor terminal device 200. The storage unit 302 stores identification information unique to its own device.

The transmission unit 303 transmits the identification information of the device received from the sensor terminal device 200 and the identification information of the relay terminal device 300 to the external terminal device 400 via the communication network NW. Note that the transmission unit 303 can also transmit the biological information of the user measured by the sensor terminal device 200.

Configuration of External Terminal Device

The external terminal device 400 includes a reception unit 401, a data analysis unit 402, a storage unit 403, and a presentation unit 404. The external terminal device 400 obtains and presents the action history of the user. Note that the data analysis unit 402 in FIG. 6 includes the first acquisition unit 10, the second acquisition unit 11, the user identification unit 12, the position identification unit 13, the action history calculation unit 14, and the interpolation unit 15 described in FIG. 1.

The external terminal device 400 is used by, for example, medical care staffs and long-term care staffs who are responsible for care of the user such as rehabilitation and treatment.

The reception unit 401 receives the identification information of the sensor terminal device 200 and the identification information of the relay terminal device 300 from the relay terminal device 300 via the communication network NW. The reception unit 401 can also receive the biological information of the user measured by the sensor terminal device 200.

The data analysis unit 402 obtains the action history of the user from the identification information of the sensor terminal device 200 and the identification information of the relay terminal device 300, and, when detecting a loss period in the action history, interpolates data of the action history from the pieces of identification information of the relay terminal device 300 immediately before and after the loss period.

The storage unit 403 corresponds to the storage unit 16 described in FIG. 1, and stores the user information and the identification information of the sensor terminal device 200 in association with each other. Further, the storage unit 403 stores the identification information of the relay terminal device 300 and the information indicating the arrangement position in the facility where the relay terminal device 300 is arranged in association with each other.

The presentation unit 404 corresponds to the presentation unit 17 described in FIG. 1. The presentation unit 404 can display the action history of each user and the biological information of the user measured by the sensor terminal device 200 on the display screen.

As described above, according to the monitoring system of the first embodiment, the action history of the user is obtained based on the identification information of the sensor terminal device 200 that identifies the user and the identification information of the relay terminal device 300 that indicates the position information of the user. Further, when the time series of the action history of the user includes a loss period, the monitoring system interpolates the action history of the user from the pieces of position information of the user immediately before and after the loss period.

Therefore, not only can the action history of the user be grasped, but also an accurate action history can be obtained by performing interpolation processing even when the data includes a loss period. As a result, it becomes possible to give more concrete and appropriate advice for improving life to the user.

For example, when it is found out from the action history of the user that the user spends most of daytime hours of the day at the same position, medical care staffs and others can advise the user to walk to a specific position in the facility in order to encourage the user to increase their amount of activity.

### Second Embodiment

Next, a second embodiment of the present invention will be described. Note that in the following description, the same components as those in the first embodiment described above are given the same reference numerals, and the description thereof will be omitted.

The first embodiment has described a case where the position information of the user in the facility is acquired and the action history of the user is obtained from the identification information and the position information of the user. In contrast, in the second embodiment, the position information of the user in the facility is given metadata indicating an attribute of the position information, and the action history of the user is obtained based on a common attribute of the pieces of position information.

Functional Blocks of Monitoring System

FIG. 7 is a block diagram showing a configuration of a monitoring system according to the second embodiment. The monitoring system includes the first acquisition unit 10, the second acquisition unit 11, the user identification unit 12, the position identification unit 13, the action history calculation unit 14, the interpolation unit 15, the storage unit 16, the presentation unit 17, and a metadata giving unit 18. The monitoring system according to this embodiment is different from that in the first embodiment in that the metadata giving unit 18 is provided. Hereinafter, configurations different from those of the first embodiment will be described mainly.

The metadata giving unit 18 gives the position information of the user acquired by the second acquisition unit 11 metadata describing an attribute representing the position information.

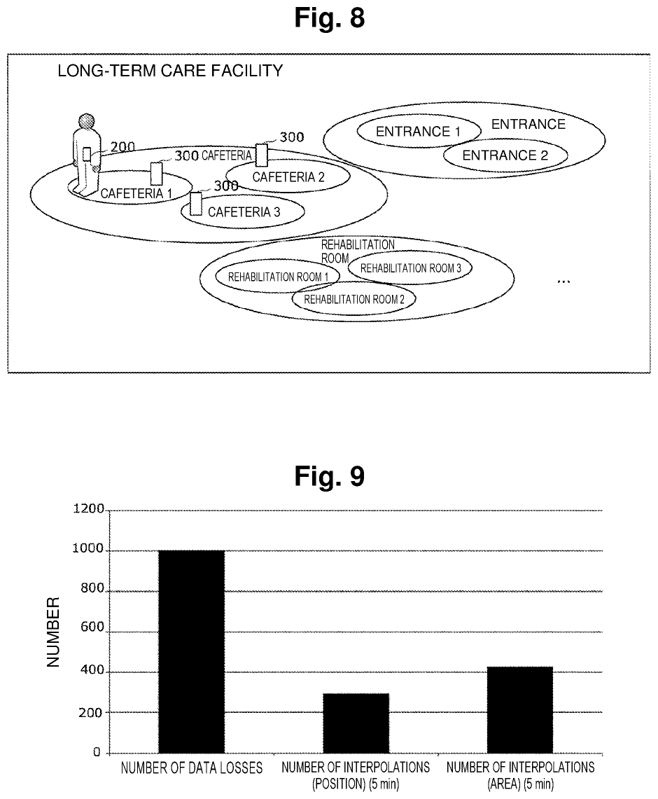

Here, an example of the metadata will be described with reference to FIG. 8. Note that FIG. 8 will be described using an example configuration in which the monitoring system includes the sensor terminal device 200, the relay terminal device 300, and the external terminal device 400 described in FIG. 5.

As shown in FIG. 8, for example, three relay terminal devices 300 are arranged at certain intervals so as to be able to cover a relatively large cafeteria area. The relay terminal devices 300 have their respective pieces of unique identification information, which identify a “cafeteria 1”, a “cafeteria 2”, and a “cafeteria 3”, indicating more detailed positions in the entire “cafeteria”.

For example, when it is desired to simply grasp the period for which and the frequency at which the user was in the cafeteria as the action history of the user, the pieces of identification information that identify the detailed positions in the cafeteria as shown in FIG. 8 have no value from the point of view of identifying only the position of the cafeteria. Therefore, when the pieces of identification information indicating the pieces of position information have a common attribute in obtaining the action history of the user, the metadata giving unit 18 gives metadata to the pieces of position information acquired by the second acquisition unit 11. In the example of FIG. 8, the three pieces of position information are given an attribute of “cafeteria” as a common attribute.

As a method for the metadata giving unit 18 to give metadata to the position information of the user, an algorithm such as clustering can be used. Alternatively, the metadata giving unit 18 can also give metadata to the position information acquired by the second acquisition unit 11 in accordance with an operational input from the outside that is received by an input device not shown.

The action history calculation unit 14 obtains the action history of the user based on the identification information unique to the user acquired by the first acquisition unit 10 and the metadata given to the position information of the user acquired by the second acquisition unit 11. Using the example in FIG. 8, even when the second acquisition unit 11 has acquired any position information of “cafeteria 1”, “cafeteria 2”, and “cafeteria 3”, the staying period and staying frequency of the user in the “cafeteria” are obtained based on the metadata “cafeteria 1” given to them.

When a loss period is detected in the time series of the action history of the user and the metadata given to the pieces of position information immediately before and after the loss period match each other, the interpolation unit 15 interpolates the action history of the user using the value of the metadata. According to the above example, even when the position information immediately before the loss period is “cafeteria 1” and the position information immediately thereafter is “cafeteria 3”, metadata “cafeteria” assigned to them match each other. Therefore, it can be considered that the user was in the “cafeteria” during the loss period.

FIG. 9 is a diagram for describing the effect of the interpolation unit 15 according to this embodiment. The bar graph on the left side of FIG. 9 shows the number of losses included in the action history data over a period of time when the interpolation processing is not performed, and shows that about 1,000 data losses have occurred. The bar graph in the middle of FIG. 9 shows the effect of the interpolation unit 15 according to the first embodiment. The bar graph in the middle of FIG. 9 shows the number of cases of interpolation processing of the action history when the interpolation processing is performed for losses that occurred for a period of 5 minutes or less over the same period of time based on more detailed position information, and the action history data is improved by about 300 pieces by the interpolation processing.

The bar graph on the right side of FIG. 9 shows the number of cases of interpolation processing of the action history when the interpolation processing is performed for losses of the action history data that occurred for a period of 5 minutes or less based on the metadata of the position information, and the action history data is improved by more than 400 pieces by the interpolation processing. As shown in FIG. 9, it can be seen that a more accurate action history of the user is obtained when the interpolation processing is performed based on the metadata given to the position information.

Although FIG. 9 shows a case where the interpolation processing is performed for data losses that occurred for a period of 5 minutes or less, the interpolation unit 15 may perform division into cases as to whether or not to perform interpolation based on, for example, the length of a loss period in the action history of the user. For example, when a loss period included in the action history is relatively long, the user may intentionally have moved out of (e.g., gone out of) the communication area covered by the relay terminal device 300.

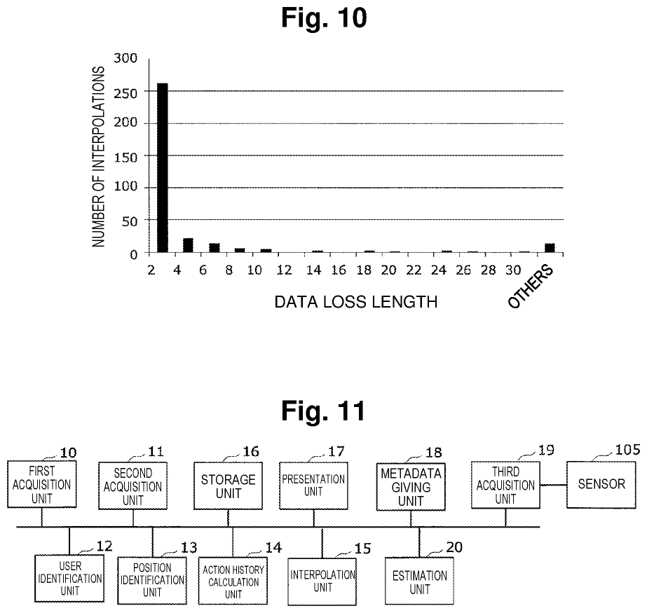

Therefore, by detecting a loss period with a length according to the user's daily life of and the level of activity amount and performing the interpolation processing of the action history, generation of an incorrect action history by the interpolation processing is prevented. For example, from the relationship between the number of cases of interpolation processing and the lengths of loss periods shown in FIG. 10, the interpolation unit 15 can determine loss periods to be subjected to interpolation processing.

As described above, according to the second embodiment, the pieces of position information of the user are given metadata representing an attribute common to the pieces of position information, and calculation and interpolation processing of the action history of the user are performed based on the metadata of the pieces of position information. Therefore, it becomes possible to more accurately grasp the action history of the user in their daily life.

### Third Embodiment

Next, a third embodiment of the present invention will be described. Note that in the following description, the same components as those in the first and second embodiments described above are given the same reference numerals, and the description thereof will be omitted.

The first and second embodiments have described a case where the time series of the action history of the user is obtained and the heart rate or the like measured by the sensor 105 attached to the user is presented together with the action history of the user. On the other hand, in the third embodiment, a specific activity performed by the user is estimated based on the biological information of the user measured by the sensor 105 and the action history of the user.

Functional Blocks of Monitoring System

FIG. 11 is a block diagram illustrating a configuration of a monitoring system according to this embodiment. The monitoring system according to this embodiment is different from those in the first and second embodiments in that it further includes a third acquisition unit 19 that acquires sensor data from the sensor 105 and an estimation unit 20 that estimates the activity of the user. Hereinafter, configurations different from those of the first and second embodiments will be described mainly.

As shown in FIG. 11, the monitoring system includes the first acquisition unit 10, the second acquisition unit 11, the user identification unit 12, the position identification unit 13, the action history calculation unit 14, the interpolation unit 15, the storage unit 16, the presentation unit 17, the metadata giving unit 18, the third acquisition unit 19, and the estimation unit 20.

The third acquisition unit 19 acquires biological information of the user from the sensor 105 including, for example, a triaxial acceleration sensor and a heart rate monitor. The biological information includes physiological information such as the heart rate and blood pressure of the user, and physical information such as the acceleration and angular velocity of the user. The third acquisition unit 19 converts the acquired analog signal into a digital signal at a predetermined sampling rate. Further, the third acquisition unit 19 can perform well known signal processing such as noise removal and amplification for an acceleration signal, an electrocardiographic signal, and the like if necessary.

The estimation unit 20 estimates the specific activity performed by the user based on the biological information of the user acquired by the third acquisition unit 19 and the action history of the user obtained by the action history calculation unit 14.

For example, when a heart rate monitor and an acceleration sensor are used as the sensor 105 and when the position of the user for a certain period according to the action history obtained by the action history calculation unit 14 was, for example, in a living room in the facility, it is assumed that the heart rate of the user exceeded a predetermined threshold (e.g., 120 [bpm]) and the state continued for 5 minutes or more. In general, it is considered that a user often rests in a living room, but, for example, in a hospital or a long-term care site, it is rather more natural to recognize that the user is getting exercise such as some kind of activity. For example, it is also possible to think that the user is performing voluntary training or recreational activity in the living room.

Therefore, the metadata of the specific activity, such as “exercise”, of the user is stored in advance in the storage unit 16. The storage unit 16 can store, for example, the position in the facility (e.g., a living room), a heart rate threshold (120 [bpm]), and the duration (e.g., 5 [minutes]) of the state in which the heart rate exceeds the threshold at that position in association with each other. The specific activity of the user is not limited to “exercise”, and it is possible to generate metadata about a desired activity of the user such as “sleep” or “walking” into which “exercise” is further classified, and store it in the storage unit 16 in advance.

The estimation unit 20 refers to the storage unit 16 to estimate the occurrence of the specific activity such as “exercise”, the period of occurrence of the specific activity, and the frequency of occurrence from the action history of the user and the biological information of the user. Using the above specific example, the estimation unit 20 estimates from the action history of the user that the user performed “exercise” for 6 minutes once in the living room when the heart rate exceeded 120 [bpm] for 6 minutes while the user is in the living room.

The presentation unit 17 displays an estimation result by the estimation unit 20 on, for example, the display screen of the display device 109.

Monitoring Method

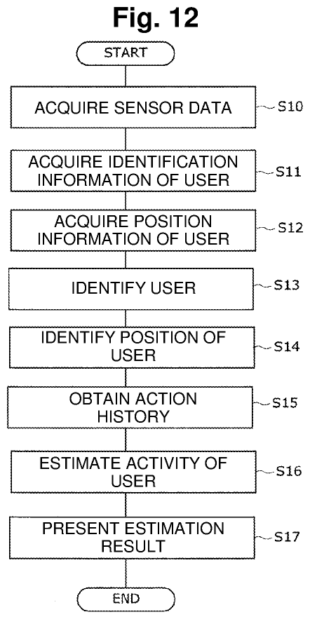

Next, operation of the monitoring system having the above-described configuration will be described using a flowchart in FIG. 12. In the following, user information (e.g., the user's name or patient ID) and unique identification information (e.g., a MAC address or an IP address) of the wearable device attached to the user are registered in the storage unit 16.

Further, the storage unit 16 stores identification information (e.g., a MAC address or an IP address) of a point arranged at a fixed position in the facility or the relay terminal device 300 and information indicating the arrangement position (e.g., the name such as “cafeteria” or “living room”) in association with each other. Furthermore, the storage unit 16 stores position information (e.g., a “living room”) and conditions such as thresholds (e.g., 120 [bpm] for 5 minutes or more) set for biological information (e.g., the heart rate) of the user in association with each other as information indicating the occurrence of the specific activity, such as “exercise”, of the user. The storage unit 16 can store a different threshold for the biological information such as the heart rate depending on the position information.

First, when the sensor 105 including a heart rate monitor and a triaxial acceleration sensor is attached to the user and measurement of the heart rate and triaxial acceleration of the user is started, the following processing is executed.

First, the third acquisition unit 19 acquires the biological information of the user from the sensor 105 (step S1n). The third acquisition unit 19 performs signal processing of the acquired biological information including the heart rate and triaxial acceleration of the user to output a time series of the biological information.

Next, the first acquisition unit 10 acquires the identification information unique to the user (step S11). Then, the second acquisition unit 11 acquires the position information of the user (step S12). For example, the second acquisition unit 11 can acquire the position information of the user at a preset cycle.

Next, the user identification unit 12 identifies the user from the identification information of the user acquired by the first acquisition unit 10 (step S13). Next, the position identification unit 13 identifies the position of the user from the position information acquired by the second acquisition unit 11 (step S14).

Next, the action history calculation unit 14 obtains the action history of the user (step S15). More specifically, the action history calculation unit 14 calculates the frequency at which and the period for which the user stayed at the identified position in the facility.

Thereafter, the estimation unit 20 estimates the specific activity performed by the user based on the action history of the user obtained in step S15 and the biological information of the user acquired in step S1n (step S16). For example, when a period of 5 minutes for which the heart rate exceeded the threshold (120 [bpm]) is detected in the period during which the user stayed in the living room, the estimation unit 20 estimates that the user performed “exercise”, which is the specific activity. Thus, the estimation unit 20 outputs an estimation result indicating that the user performed “exercise” for 5 minutes once.

The estimation unit 20 can also estimate that the user performed the specific activity based not only on the biological information such as the heart rate but also on the acceleration of the user measured by the triaxial acceleration sensor, for example. Hereinafter, a case will be described as an example where it is estimated that the user performed the specific activity based on the acceleration of the user and the action history of the user.

The estimation unit 20 obtains the average value or standard deviation per unit time of the acceleration amplitudes in the three axes of the user or the norm of the acceleration values in the three axes acquired by the third acquisition unit 19 from the sensor 105 including the triaxial acceleration sensor as body motion, and, when these values have exceeded a set threshold, estimates, for example, that the user is performing “exercise”. In this case, the storage unit 16 stores the position information in the facility, the magnitude of the body motion of the user, and the estimated activity such as “exercise” or an activity into which “exercise” is further classified in association with each other. For example, it is possible to use “mild exercise”, “moderate exercise”, and “intense exercise”, into which “exercise” is classified by levels according to the magnitude of body motion.

Further, even when the body motions having the same magnitude are calculated, the actual activities of the user may differ depending on whether the user is in the rehabilitation room or the washroom. For example, even in a case where “intense exercise” is estimated from the value of the body motion when the position of the user is in the rehabilitation room according to the action history of the user, “the possibility of falling down” can be estimated when the position of the user is in the washroom.

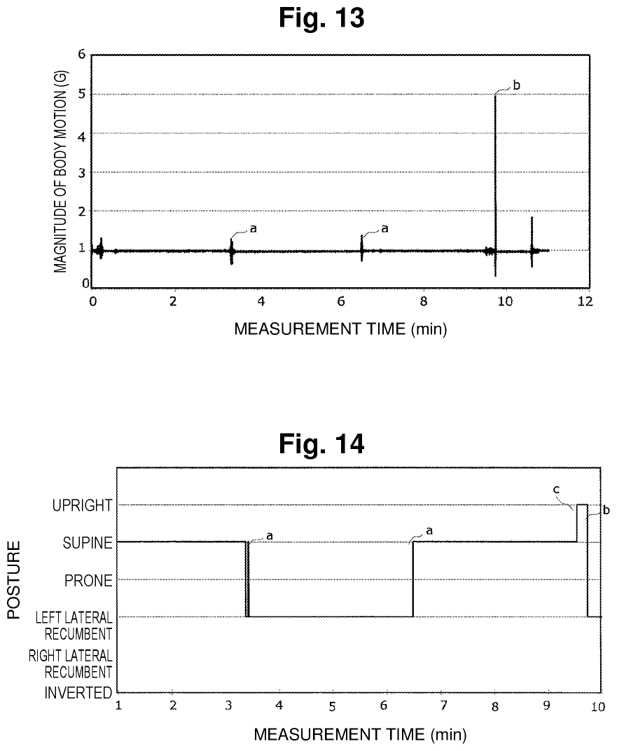

For example, FIG. 13 shows the magnitude [G] of body motion of the user at the measurement time. The example in FIG. 13 shows body motions corresponding to activities of the user that occurred while the user was lying in bed. In the example of FIG. 13, a body motion at about 1.5 [G] is measured for rolling over, and a body motion at about 5 [G] is measured for falling from the bed. For example, when a body motion exceeding 5 [G] occurred once while the user was sleeping in the hospital room, even if a body motion equivalent to “exercise” occurred, it is presumed that exercise contrary to the intention of the user, such as falling from the bed, occurred rather than “exercise” from their own will.

In this way, the estimation unit 20 estimates that the user performed the specific activity and its frequency and period based on the position information of the user and the magnitude of body motion. Further, the estimation unit 20 may make an estimation in consideration of the user's life at night and in the daytime by further using time information measured by the clock 107.

To give another example, the estimation unit 20 can calculate the user's posture from the accelerations in the three axes of the user, and estimate that the user is performing the specific activity from the action history of the user and a change in the posture. More specifically, the sensor 105 measures the accelerations in three directions along the XYZ axes that are orthogonal to each other, as shown in FIG. 5. The third acquisition unit 19 acquires the accelerations measured by the sensor 105 at a sampling rate of, for example, 25 Hz to obtain time series of accelerations.

The estimation unit 20 calculates the user's posture from the accelerations in the three axes of the user acquired by the third acquisition unit 19. More specifically, the estimation unit 20 obtains the angle of tilt of the user's upper body from the accelerations of the user. The estimation unit 20 calculates, for example, the tilts θ and ϕ [degrees] of the sensor 105 on the accelerations with respect to the gravitational acceleration, as disclosed in Reference 1 (International Publication No. WO 2018139398). Here, θ (−90≤θ<270) is the tilt of the Z-axis of the sensor 105 with respect to the vertical direction, and ϕ (−90≤ϕ<270) is the tilt of the X-axis of the sensor 105 with respect to the vertical direction.

$$Expressions ( 1 ) and ( 2 )$$

$$θ = \frac{180}{π} {cos}^{- 1} ( \frac{{A}_{z}}{\sqrt{{A}_{x}^{2} + {A}_{y}^{2} + {A}_{z}^{2}}} ) + 90 … ( for {A}_{y} \geq 0 ) \\ θ = - \frac{180}{π} {cos}^{- 1} ( \frac{{A}_{z}}{\sqrt{{A}_{x}^{2} + {A}_{y}^{2} + {A}_{z}^{2}}} ) + 90 … ( for Ay < 0 ) \qquad ( 1 )$$

$$ϕ = \frac{180}{π} {cos}^{- 1} ( \frac{{A}_{x}}{\sqrt{{A}_{x}^{2} + {A}_{y}^{2} + {A}_{z}^{2}}} ) + 90 … ( for {A}_{y} \geq 0 ) \\ ϕ = \frac{180}{π} {cos}^{- 1} ( \frac{{A}_{x}}{\sqrt{{A}_{x}^{2} + {A}_{y}^{2} + {A}_{z}^{2}}} ) + 90 … ( for Ay < 0 ) \qquad ( 2 )$$

Ax, Ay, and Az are the accelerations in the X, Y, and Z-axis directions measured by the sensor 105, respectively, and the unit is the gravitational acceleration G (1.0 G≈9.8 m/s²). In Expressions (1) and (2), by obtaining the ratio of the measured value in a single axis with respect to the norm, which is the magnitude of the composite vector of the accelerations in the X, Y, and Z axis directions measured by the sensor 105, and further obtaining the inverse function of the cosine, the tilt of the sensor 105 (the sensor terminal device 200 in FIG. 5) is calculated as a value having the dimension of angle.

The estimation unit 20 determines the user's posture from the obtained tilts of the sensor 105. For example, the estimation unit 20 determines the posture by comparing the values of θ and ϕ calculated by Expressions (1) and (2) with thresholds. The tilt of the sensor 105 reflects the tilt of the upper body of the user wearing the sensor terminal device 200 (the sensor 105) equipped with the sensor 105.

The estimation unit 20 can determine the user's posture using the division of the ranges of values of θ and ϕ into cases described in Reference 1. Specifically, the values of θ and ϕ are classified so that the user's posture is classified into six types: upright, inverted, supine, prone, right lateral recumbent, and left lateral recumbent. For example, the estimation unit 20 determines that the user is in supine posture when [130≤ϕ≤230] and [−40≤θ<30], or when [1.30≤ϕ≤230] and [140<θ<220].

Further, the estimation unit 20 determines that the user's posture is upright when [30≤θ<140].

Alternatively, the estimation unit 20 can also determine the user's posture by classifying the values of θ and ϕ into two types: a wake-up state and a lying-down state.

FIG. 14 is a diagram showing changes in posture when the user's posture is classified into six types. FIG. 14 shows changes in posture when the user is lying on the bed and resting, and “a” indicates a change in posture when the user rolled over. “b” indicates a change in posture when the user fell from the bed, and “c” indicates a change in posture when the user performed a rising action.

As shown in FIG. 14, when the user performs a rising action (“c” in FIG. 14), the posture transitions from supine to upright. On the other hand, at the time of rolling over (“a” in FIG. 14), it transitions from supine to prone or from prone to supine. From this, it is possible to distinguish and estimate a specific motion of the user such as rolling over or rising based on the change pattern of the posture.

The estimation unit 20 estimates that the user performed a specific action when the change in the user's posture has a set change pattern. Furthermore, when the change in the user's posture became the change pattern of a specific posture at a specific position at a certain frequency according to the action history of the user, the estimation unit 20 estimates the occurrence of a particular exercise corresponding to the change pattern of the posture and its period and frequency.

For example, when the user's posture has changed from supine to upright ten times in the rehabilitation room, the estimation unit 20 can estimate that the user is performing “rehabilitation exercise” in the rehabilitation room, and can further output the period and frequency of occurrence of the changes in posture.

In this way, the estimation unit 20 estimates that the user performed the specific activity based on the biological information of the user and the position information of the user.

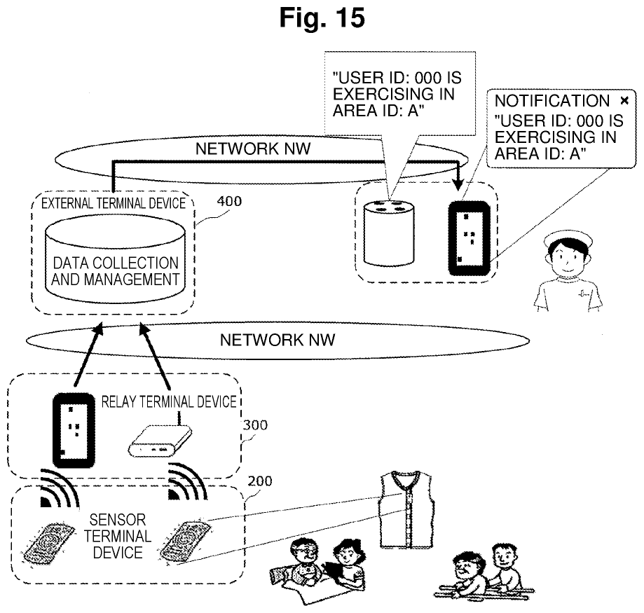

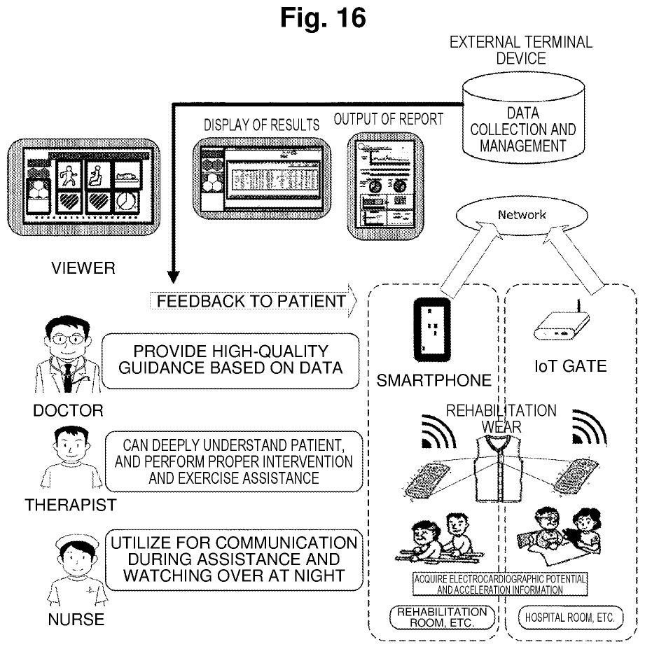

FIG. 15 is a diagram showing the entire monitoring system according to this embodiment, which includes the sensor terminal device 200 implemented by a wearable device attached to the user, the relay terminal device 300, and the external terminal device 400. The relay terminal device 300 receives the biological information of the user and the identification information unique to the sensor terminal device 200 from the sensor terminal device 200, and transmits them to the external terminal device 400. The external terminal device 400 receives the identification information of the relay terminal device 300, the biological information of the user, and the identification information of the sensor terminal device 200 from the relay terminal device 300 via the communication network NW, and estimates the action history of the user and the activity of the user.

The specific activity and the action history of the user estimated by the external terminal device 400 can be presented to, for example, a communication terminal device such as an external smart speaker or a smartphone. As shown in FIG. 15, in a medical care facility and a long-term care facility, medical care staffs and long-term care staffs responsible for the treatment and care for the user can grasp the estimated user's activity and action history. From the estimated user's activity and user's action history, the medical care staffs and long-term care staffs can give more concrete and appropriate guidance for improving their life when trying to increase the amount of activity of the user, for example.

As described above, according to the third embodiment, it is estimated that the user performed the specific activity based on the biological information of the user measured by the sensor 105 and the action history of the user. For example, it is possible not only to estimate occurrence of the specific activity, which is more likely to occur when the user stays in a room where the specific activity is performed such as a rehabilitation room, but also to estimate that the user is performing the specific activity such as exercise even in a place where exercise is not performed originally.

Note that in the third embodiment described above as well, the interpolation unit 15 can perform the interpolation processing of the action history. Furthermore, the action history can be obtained based on the metadata given to the position information by the metadata giving unit 18.

Further, the above-described embodiments have illustrated and described cases where one sensor terminal device 200 is provided. However, there may be a plurality of users.

Although the monitoring system, the monitoring method, and the monitoring program of embodiments of the present invention have been described above, the present invention is not limited to the described embodiments, and it is possible to make various modifications that can be envisaged by those skilled in the art within the scope of the invention described in the claims. For example, the first to third embodiments described above can be implemented in any combination. Further, the order of the steps of the monitoring method is not limited to the order described above.

### REFERENCE SIGNS LIST

10 First acquisition unit
11 Second acquisition unit
12 User identification unit
13 Position identification unit
14 Action history calculation unit
15 Interpolation unit
16, 203, 302, 403 Storage unit
17, 404 Presentation unit, data analysis unit
12, 304 Imaging control unit
13, 402 Imaging data acquisition unit
101 Bus
102 Processor
103 Main storage device
104 Communication I/F
105, 201 Sensor
106 Auxiliary storage device
107 Clock
108 Input/output I/O
109 Display device
200 Sensor terminal device
202 Sensor data acquisition unit
300 Relay terminal device
400 External terminal device
204, 303, 404 Transmission unit
301, 401 Reception unit
402 Data analysis unit

1-8. (canceled)

9. A monitoring system comprising:
a first acquirer configured to acquire identification information unique to a user;
a second acquirer configured to acquire position information of the user;
a calculator configured to obtain an action history of the user from the identification information of the user acquired by the first acquirer and the position information acquired by the second acquirer; and
a presenter configured to present the action history of the user calculated by the calculator, wherein the action history comprises a period for which or a frequency at which the user stayed at a position indicated by the position information.

10. The monitoring system according to claim 9, further comprising an interpolator configured to, when a loss period of data is included in the action history of the user obtained by the calculator and the position information of the user immediately before the loss period matches the position information of the user immediately after the loss period, interpolate the action history of the user including the loss period based on the matched position information.

11. The monitoring system according to claim 10, wherein the presenter is configured to present the action history of the user interpolated by the interpolator.

12. The monitoring system according to claim 9, further comprising a metadata provider configured to give metadata describing an attribute representing the position information to the position information, wherein the calculator is configured to calculate the action history of the user based on the identification information of the user acquired by the first acquirer and the metadata given to the position information acquired by the second acquirer.

13. The monitoring system according to claim 9, further comprising:
a sensor data acquirer is configured to acquire biological information of the user; and
an estimator configured to estimate a specific activity performed by the user based on the acquired biological information and the action history of the user.

14. The monitoring system according to claim 13, wherein the presenter is configured to present an estimation result by the estimator.

15. A monitoring system comprising:
a sensor terminal device configured to be attached to a user and to output first identification information to outside, wherein the first identification information is unique to the sensor terminal device;
a relay terminal device configured to be arranged at a predetermined position within an area, to receive the first identification information output from the sensor terminal device, and to output the first identification information and second identification information to the outside, wherein the second identification information is unique to the relay terminal device; and
an external terminal device configured to receive the first identification information and the second identification information output from the relay terminal device and to store the first identification information and the second identification information in a storage device, wherein the external terminal device comprises:
a first acquirer configured to acquire the first identification information as identification information unique to the user;
a second acquirer configured to acquire the second identification information as position information of the user;
a calculator configured to obtain an action history of the user from the identification information of the user acquired by the first acquirer and the position information acquired by the second acquirer; and
a presenter configured to present the action history of the user obtained by the calculator, wherein the action history comprises at least one of a period for which or a frequency at which the user stayed at a position indicated by the position information.

16. A monitoring method comprising:
acquiring identification information unique to a user;
acquiring position information of the user;
obtaining an action history of the user from the identification information of the user and the position information; and
presenting the action history of the user, wherein the action history comprises at least one of a period for which or a frequency at which the user stayed at a position indicated by the position information.

17. The monitoring method according to claim 16, further comprising, when a loss period of data is included in the action history of the user and the position information of the user immediately before the loss period matches the position information of the user immediately after the loss period, interpolating the action history of the user including the loss period based on the matched position information.

18. The monitoring method according to claim 17, wherein presenting the action history of the user comprises presenting the interpolated action history of the user.

19. A monitoring program for causing a computer to execute the monitoring method according to claim 16.
